(19) United States
(12) Patent Application Publication
(10) Pub. No.: US 2023/0326577 A1
(43) Pub. Date: Oct. 12, 2023

(54) ARTIFICIAL INTELLIGENCE MENTAL HEALTH DIAGNOSTIC SYSTEM AND METHOD

(71) Applicant: Ben Prince, West Hartford, CT (US)

(72) Inventors: Ben Prince, West Hartford, CT (US); Evan Greebel, Scarsdale, NY (US); Travis James, Frisco, TX (US)

(21) Appl. No.: 18/299,346

(22) Filed: Apr. 12, 2023

Related U.S. Application Data
(60) Provisional application No. 63/330,019, filed on Apr. 12, 2022.

(51) Int. Cl. G16H 20/70; G16H 20/10; G16H 80/00; G16H 10/20

(52) U.S. Cl. CPC G16H 20/70; G16H 10/20; G16H 80/00; G16H 20/10

(57) ABSTRACT

A method uses a machine learning model to recommend a course of mental health treatment by a health platform. The health platform ingests a first data set from a first medical diagnostics assessment of a patient and a second data set of identifying factors associated with the patient. A machine learning model is applied to the first and second data sets and a course of mental health treatment is generated. The course of mental health treatment is displayed within a graphical user interface. The health platform can also include ingesting a third data set from a second medical diagnostics assessment of the patient and receiving data related to the third data set from a big data source. The health platform applies the machine learning model to the third data set and the data from the big data source and updates the generated course of mental health treatment.

### STATEMENT OF RELATED INVENTIONS

This application is a U.S. Nonprovisional application which claims the benefit of U.S. Provisional Application No. 63/330,019, filed Apr. 12, 2022, and is hereby incorporated by reference in its entirety.

### SUMMARY

The below summary is merely representative and non-limiting.

In a first aspect, an embodiment provides a method of using a machine learning model to recommend a course of mental health treatment. The method includes ingesting (for example, by processing, analyzing, etc.), by a health platform, a first data set from a first medical diagnostics assessment of a patient. The health platform ingests a second data set of identifying factors associated with the patient. The method includes applying, by the health platform, to the ingested first and second data sets, a machine learning model and generating, based on the applying the machine learning model to the first and second data sets, a course of mental health treatment. The generated course of mental health treatment is displayed within a graphical user interface.

The method can also include ingesting, by the health platform, a third data set from a second medical diagnostics assessment of the patient and receiving, by the health platform, from a big data source, data related to the third data set. The health platform applies the machine learning model to the third data set and the data from the big data source; and updates the generated course of mental health treatment.

In another aspect, an embodiment provides a computer readable medium tangibly encoded with a computer program to recommend a course of mental health treatment. The computer program is executable by a processor to perform actions. The actions include ingesting a first data set from a first medical diagnostics assessment of a patient and ingesting a second data set of identifying factors associated with the patient. The actions also include applying to the ingested first and second data sets, a machine learning model and generating, based on the applying the machine learning model to the first and second data sets, a course of mental health treatment. A graphical user interface is used to display the generated course of mental health treatment.

### BRIEF DESCRIPTION OF THE FIGURES

FIG. 1 is a functional block diagram illustrating an example computing architecture, in an embodiment.

FIG. 2A is a flowchart of a process for implementing a workflow for connecting a patient with a mental-health provider based on a health assessment, in an embodiment.

FIG. 2B is a continuation of the flowchart of the process for implementing the workflow for connecting the patient with a mental health provider based on the health assessment, in an embodiment.

FIG. 2C is a continuation of the flowchart of the process for implementing the workflow for connecting the patient with a mental health provider based on the health assessment, in an embodiment.

FIG. 3 is a flowchart illustrating an example method of a system generating a recommendation based on ingesting first and second sets of data, in an embodiment.

FIG. 4 is a functional block diagram illustrating an example computing architecture that includes components from FIG. 1, in an embodiment.

FIG. 5 is an example machine learning model used by the rules engine for generating, based on ingested data sets and big data source, in an embodiment.

FIG. 6 is a diagram of an example environment in which systems and/or methods described herein may be implemented, in an embodiment.

### DETAILED DESCRIPTION OF THE EMBODIMENTS

Embodiments of the present disclosure include a comprehensive platform for supporting mental healthcare and further for connecting a patient with a mental-health provider. Typically, a patient can fill out a questionnaire that may indicate a particular type of mental illness or state based on the answers provided by the patient. Based on the results of the questionnaire, a health care provider or someone trained to interpret the results of the questionnaire may determine a prescription or course of treatment for the patient to overcome a condition. However, a health care provider may introduce a conscious or subconscious bias (e.g., based on their experience, training, specialty, etc.) that leads to an imperfect prescription or course of treatment for the patient.

Advantageously, present embodiments remove biases of a healthcare provider, by introducing an artificial intelligence (AI) system (e.g., machine learning techniques) that ingests the questionnaire and, neutrally, recommends a healthcare provider to contact the patient, identifies a potential course of treatment, and/or a possible appropriate prescription. In making the recommendations, the system may consider identifying factors of the patient, including, but not limited to, income, age, gender, sexual orientation, race, education level, military experience, etc. For example, the system may match a patient to a mental health provider best suited for the patient's assessment results; match a patient to a self-care regimen based on the assessment results; and/or match a patient to a coach or other ecosystem provider suited to help based on the results. The system may also identify potential courses of treatment to be considered and implemented. The resulting output may recommend the types of care and frequency of care, e.g., therapy or meeting with a psychiatrist or similarly trained clinician. The results may then be examined by an appropriate clinician. The system may compare the resulting output to recommendations made by healthcare providers and learn from the difference in recommendation between the outputted results and the recommendations made by healthcare providers. In addition, the system provides ongoing treatment, e.g., by having monthly, quarterly, or yearly assessments that are analyzed against historical assessments. The system learns from this analysis to improve on outputted results that take into consideration patient progress or lack thereof.

The present embodiments further provide a set of tools to make mental health assessments available to the patient (e.g., via mobile application or web portal), so data can be collected to further guide the mental health workflow (e.g., direct the patient to the resources most applicable to them based on MINI and other assessment results). Further, the present embodiments provide a secure, HIPAA-compliant data-sharing pipeline for sharing assessment and other healthcare data to providers with the consent of the patient. Embodiments further provide a healthcare provider portal where doctors can retrieve patient data from assessments, other providers, etc. in a HIPAA-compliant manner to assist in providing the best mental health care.

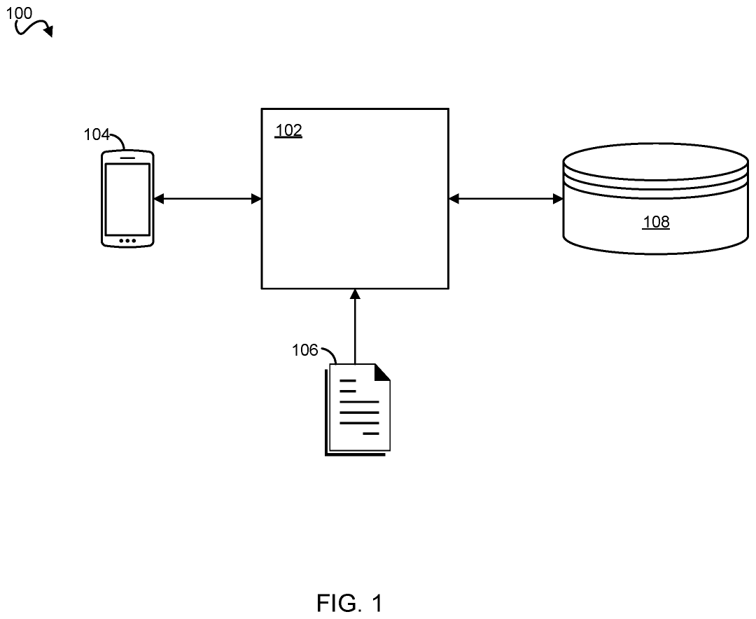

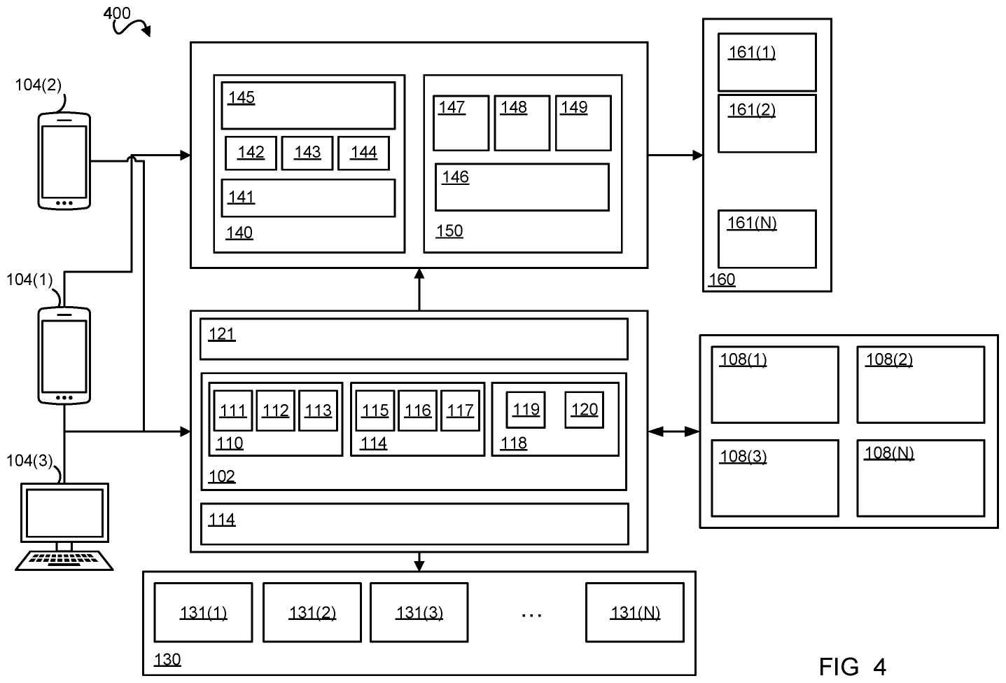

The following diagram shown in FIG. 1 illustrates a system 100 that provides a framework for primary care physicians to direct patients who are candidates for mental healthcare to refer to the mental health platform 102. A patient or a health care provider may access the mental health platform 102 remotely via a client application running on a client device 104 or by a portal (not shown) designed for healthcare providers. The platform 102 may receive a survey (e.g., a survey, medical diagnostics test, etc.) from a patient using the client device 104 running the client application. The client application may integrate with one or more medical diagnostics platforms (e.g., the diagnostics platforms 131(1)-(N) in FIG. 4).

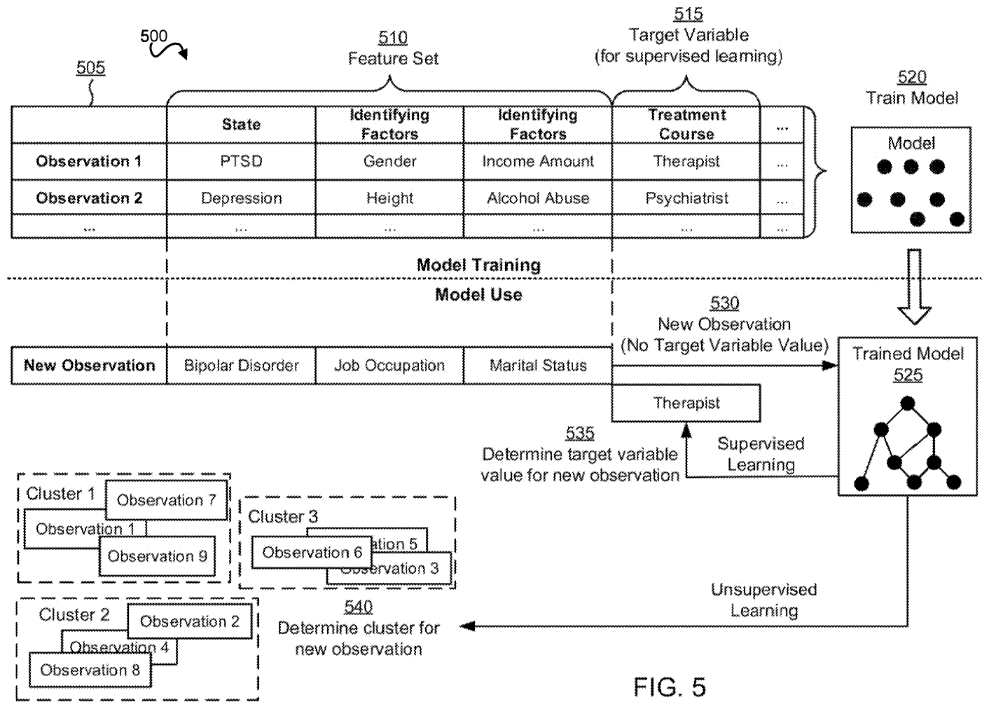

Further, the platform 102 may receive identifying information about the patient from external sources 106 (e.g., from medical records, self-assessment, etc.) or from the client device 104 inputted by the patient. In one example, the identifying information may be a non-exhaustive list, including age, gender, medical history (e.g., mental health, fitness, past injuries, surgeries, diseases, etc.), education, marital status, income, alcohol and narcotics history, sexual orientation, race, ethnicity, height, weight, etc. The health platform 102 may ingest both the first data set (the diagnostics test) and the second data set (the identifying information), input the data sets into a machine learning model (e.g., the machine learning system 500 in FIG. 5), and generate a recommendation for the patient. In some embodiments, the machine learning model may incorporate historical data from big data 108, which may include previous recommendations and the data used to generate those recommendations. The components illustrated in FIG. 1 are illustrated in more detail in FIGS. 4 and 5.

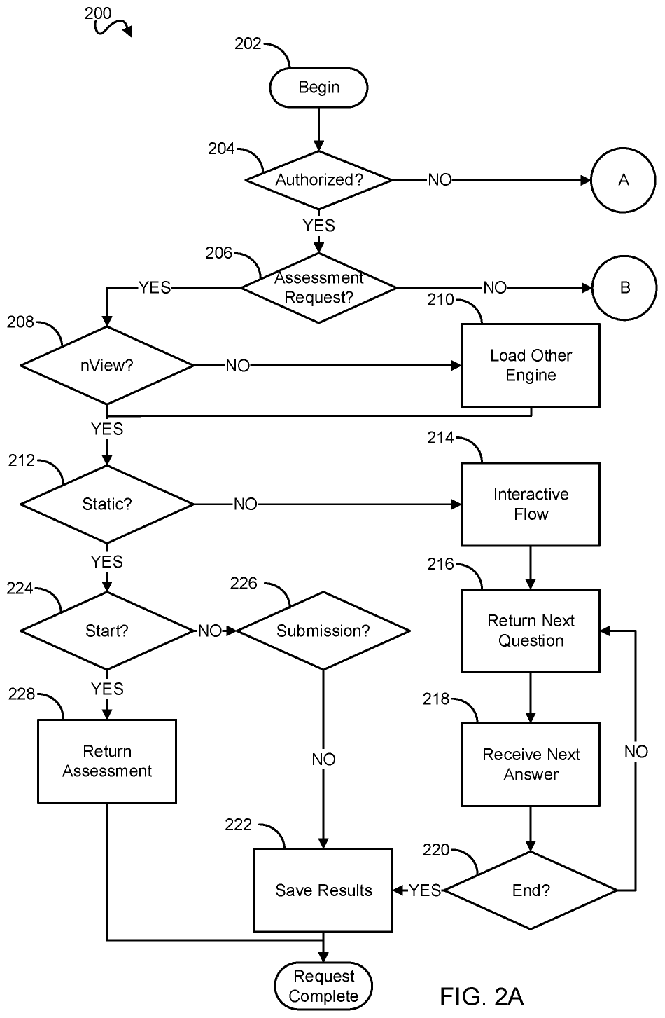

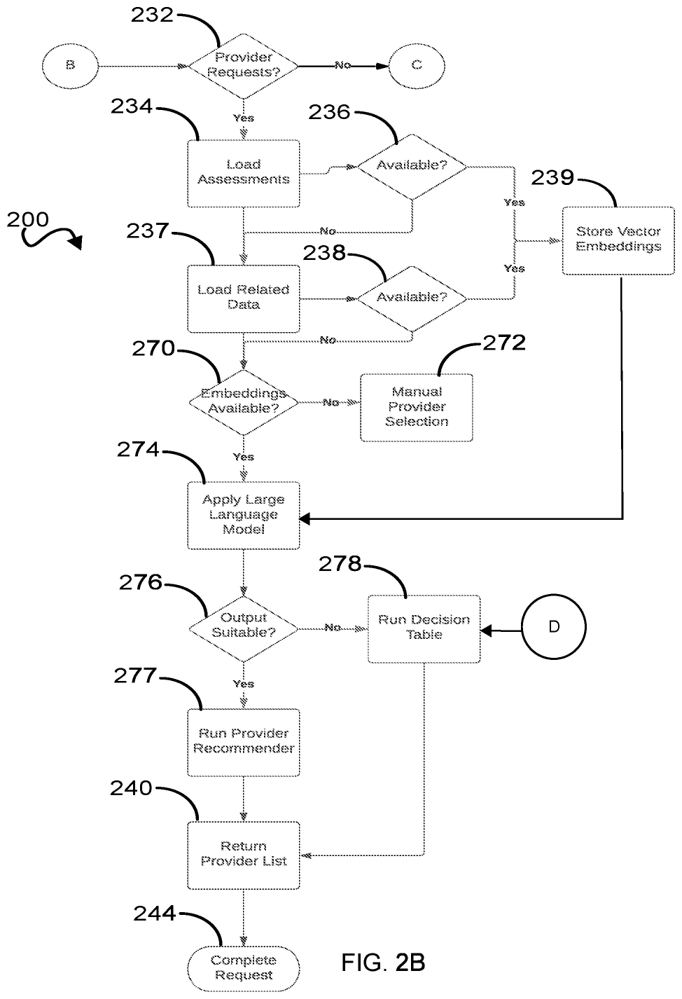

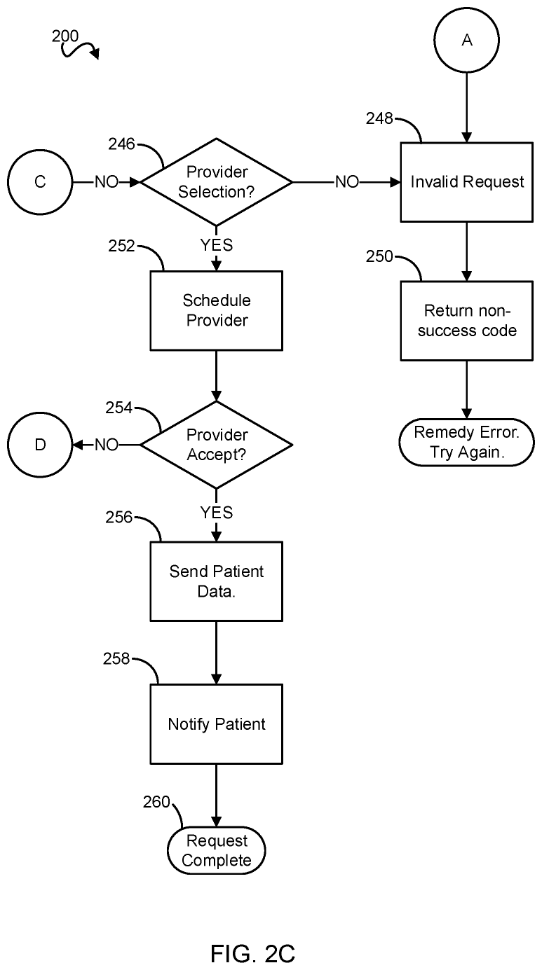

FIG. 2A is a flow chart illustrating an example method 200 of the system ingesting a data set to generate a recommendation for a patient. When method 200 begins (202), it determines (204) whether a request is authorized, e.g., by a patient recommended by a primary care physician associated with the present embodiments. At block 204, if the system determines (decision branch: “YES”) that the patient is authorized, the system determines (206) whether the request is an assessment request. For example, whether the system will ingest a data set relating to a medical diagnostics assessment from a medical diagnostics platform (e.g., the medical diagnostics platforms 131(1)-(N)), such as nView © or any other medical diagnostics service. Method 200 may include the latest planned version of the nView © assessment API, which allows for the embedding of assessments in any container (not just via I-frame), allowing the system to collect data in real-time and store it in a manner that the system can be run in order to identify the best providers; however, any medical diagnostics test or application is within the scope of the present embodiments.

However, if the system determines, at block 204, the request is not authorized (decision branch: “NO”), method 200 will proceed to step A, with reference to FIG. 2C. At block 208, the system determines the type of medical diagnostics engine being used (e.g., nView ©, etc.). At block 208, if the system determines (decision: “NO”) the medical diagnostics engine being used to conduct the mental health assessment is not nView ©, the system will proceed to load (210) another engine. In one example of 210, the system loads another engine that is compatible with the medical diagnostics test the patient is using. Once the engine is loaded, the system returns to the main flow. Method 200 further includes the system determining (212) if the medical diagnostics test is static (e.g., if the patient is submitting a completed medical diagnostics assessment). In one example of decision block 212, the system 100 makes a distinction between static and interactive assessments. A static assessment is an assessment where all the questions are known in advance and can be returned to a caller of the system in one request. In this case, answers are submitted in a single request at the end of the assessment.

An interactive assessment is where the question flow is determined by a previous user response to a previous question. In some embodiments, a following question is based on an answer to a previous question. For example, the following question is not determined until the user has submitted an answer to the previous question. In this case, answers are submitted one at a time as part of a long-running workflow (214)-(220) until the last question is answered. Once the last question is answered, the results are processed and saved (222) for future reference. The health platform 102 ensures that a user can stop, start, and continue an interactive session. If the health platform 102 determines the medical diagnostic assessment is interactive (decision block 212: “NO”), the system proceeds to implement (214) where the interactive flow of the interactive assessment begins, including returning (216) a next question for the user to answer and receiving (218) a next answer until the interactive assessment ends (220). Once the interactive assessment ends, the results of the assessment are saved (222), completing the request. In one example, the health platform 102 saves the results of the interactive assessment to big data 108.

At block 212, if the health platform 102 determines (212) the assessment is static (decision: “YES”), the health platform 102 determines whether to start (224) the assessment. If the health platform 102 starts (decision: “YES”), the health platform 102 returns the assessment, completing the request. However, if the health platform 102 does not start (decision: “NO”), the health platform 102 determines there is no submission and saves (222) the results, completing the request.

FIG. 2B is a continuation flowchart illustrating the method 200 of the health platform 102 ingesting a data set to generate a recommendation for a patient. At block B, the health platform 102 determines (206) (decision: “NO”) the request is not an assessment request. The health platform 102 then determines (232) whether the request is a provider request. However, if the health platform 102 determines, at block 232, the request is not a request associated with a provider authorized by the system (decision: “NO”), method 200 continues to block C, continued in FIG. 2C.

If the health platform 102 determines (232) (decision: “YES”) the request is a provider request from an authorized provider, the health platform 102 loads (234) assessments and loads (238) related data. After the health platform 102 loads the assessments, if any, it determines (236) whether any are available. The assessment (e.g., the static assessment or interactive assessment) is the saved assessment completed by the user, with reference to FIG. 2A. If assessments are available (decision: “YES”), the health platform 102 stores the assessment as a vector embedding. The embeddings represent the relatedness of the data by associating “distances” between vector elements, with shorter distances indicating high relatedness. Embeddings are attached to the patient, and a patient may have any number of embeddings from previous encounters, etc.

If, at block 236, the health platform 102 determines there are no assessments available (decision: “NO”), the health platform 102 may proceed to load (238) related data. Alternatively, the health platform 102 may load (238) related data in parallel with loading (234) assessments. The related data includes information that may come from anywhere that can be associated with a patient. Once related data is loaded, the health platform 102 determines (239) whether any are available. If related data is available (decision: “YES”), the health platform 102 stores the related data as a vector embedding. On the other hand, if the health platform 102 determines no related data is available (decision: “NO”), the health platform 102 may proceed to the next step of the process.

The health platform 102 determines if any embeddings are available (270). If none are available (decision: “NO”), the health platform 102 reverts to a “Manual Provider Selection.” (272). This may be a basic decision table that assigns a provider based solely on criteria like who is available, etc.

If embeddings are available (decision: “YES”), either stored previously or once vector embeddings are stored at step 239, the health platform 102 applies a large language model (274) to the embeddings, as one, non-limiting example, the large language model (LLM) may be GPT-4. The large language model returns content that is an enhanced assessment as well as data.

The health platform 102 determines if the output from the large language model is suitable (276). If the output is suitable (decision: “YES”), the health platform 102 runs a provider recommender (277). The recommender system knows about the various providers (their credentials, history, etc.). This may be another LLM that is fine-tuned on practitioner data. As one, non-limiting example, this may be done with a call to GPT-4 with practitioner embeddings attached to the data output from step 274). The recommender system returns a list of providers that match. In some embodiments, the list may include reasons for the match, for example, to enable further training.

The recommender system of the health platform 102 matches patients to any of the following mental health workflows: (i) match a patient to the mental health provider best suited for the patient's assessment results; (ii) match a patient to a self-care regimen based on assessment results; (iii) match a patient to a coach or other ecosystem provider suited to help based on the results. Any additional data including changes to the patient assessment and results from the recommended treatments may be added to big data 108 and further analyzed, resulting in refinement and improvement of the AI engine (e.g., machine learning model 500) over time.

Creating a set of rules for the recommender system to apply to embeddings to guide treatment may require collaboration between designers of the medical diagnostics assessment (e.g., nView ©) that the health platform 102 receives and designers of the health platform 102. The clinical logic may be a collaboration with the rules executed in one of two ways: a basic rules engine (that hasn't ingested any data sets), then followed by the evolution of an AI neural network (or machine learning model) for advanced future cases that combine the use of a wide variety of other patient data sets collected by applications over time. For example, the rules engine, over time, as the machine learning model ingests more data sets, and compares those data sets to historical data sets stored within big data 108, can learn and evolve to generate more refined, learned recommendations, as discussed with reference to FIG. 5. Once the health platform 102 has applied the rules engine, at block 242, the health platform 102 returns (240) the provider list, completing the request.

If the output is not suitable to run a recommender (decision: “NO”), the health platform 102 may run a basic decision table (278) similar to the “Manual Provider Selection” in step 272. Method 200 further continues with the health platform 102 returning (240) the provider list, completing the request (244). The provider list includes those providers identified as suitable for the request, for example, those selected by the recommender or those indicated in the decision table.

FIG. 2C is a continuation flowchart illustrating the method 200 of the health platform 102 ingesting a data set to generate a recommendation for a patient. At block A, method 200 may continue, from block 204, where the health platform 102 determined (decision: “NO”) the request was not authorized. Method 200 continues with the health platform 102 determining (248) the request is invalid and return (250) a non-success code.

At block C, method 200 may continue, from block 232, where the health platform 102 determined (decision: “NO”) the request is not from an associated provider. The health platform 102 may schedule (252) the provider and determine (254) whether to accept the provider. If, at block 256, the health platform 102 accepts (decision: “YES”) the provider, the health platform 102 sends (256) the patient data, and then notifies (258) the patient, completing the request. However, if the health platform 102, at decision block 254, does not accept the provider (decision: “NO”), method 200 continues to block D, with reference to FIG. 2B, where the health platform 102 runs a decision table (278) and returns a list of providers 240.

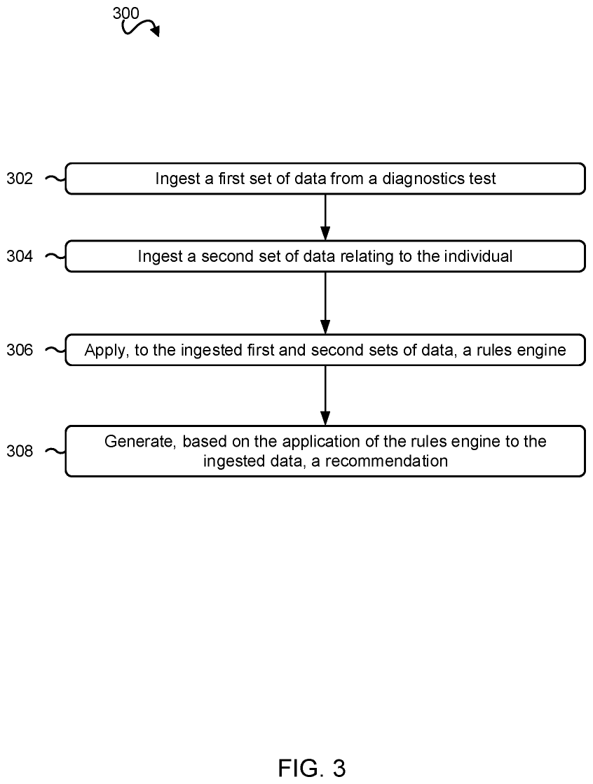

FIG. 3 is a flowchart illustrating a method 300 of generating a recommendation for a patient using a machine learning model (e.g., the machine learning model 500). Method 300 includes ingesting (302) a first data set. In one example of block 302, the first data set may be a static or interactive medical diagnostics assessment, as discussed with reference to FIG. 2A. For example, the health platform 102 ingests the data received by the medical diagnostics application (e.g., medical diagnostics application 131(1)-(N) running on the client device 104). Method 300 further includes the health platform 102 ingesting (304) a second data set. In one example of block 304, the health platform 102 ingests data from external source 106 and/or from big data 108. In one example of block 304, the health platform 102 receives identifying information of the patient, including age, gender, medical history (e.g., mental health, fitness, past injuries, surgeries, diseases, etc.), education, marital status, income, alcohol and narcotics history, sexual orientation, race, ethnicity, height, weight, etc.

Method 300 further includes health platform 102 applying (306) the rules engine (with reference to FIGS. 2B) to the first and second ingested data sets. In one example of block 306, the health platform 102 applying the rules engine includes utilizing machine learning techniques (e.g., artificial intelligence, neural networks, natural language processing, etc.) on the first and second data sets, with reference to FIGS. 4 and 5. In one example of block 306, the health platform 102 uses a machine learning model to incorporate the historical data (e.g., ingested data from the patient, including identifying information and historical static and interactive assessments) stored in big data 108 in the application of the rules engine.

For example, the rules engine ingests the first data set (e.g., the medical diagnostic assessment) to determine at least one particular state (e.g., PTSD, anxiety, depression, etc.) of the patient and a corresponding level of severity based on a numerical scale (e.g., 1 to 7). The rules engine may consider the at least one particular state in the context of the second data set. For example, the identifying information may provide more context of the at least one particular state to determine a more holistic recommended treatment. For example, if the at least one particular state is anxiety and the level of severity is a numerical value of 2, and the identifying information indicates the patient was in combat during a military deployment, the health platform 102 may generate a recommended treatment of therapy, instead of recommending a psychiatrist, who may prescribe medication as a treatment. However, if the medical diagnostic assessment results for the patient indicates a particular state of depression with a severity level of 7, and the identifying information includes a history of self-harm, resulting in hospitalization, the health platform 102 may generate a recommended treatment of psychiatry. The application of the rules engine via a machine learning model 500 is discussed in greater detail in FIG. 5.

Method 300 further includes the health platform 102 generating (308) a treatment recommendation for the patient. In one example of block 308, the treatment recommendation includes one of matching patients to any of the following mental health workflows: (i) matching a patient to the mental health provider best suited for the patient's assessment results; (ii) matching a patient to a self-care regimen based on assessment results; (iii) matching a patient to a coach or other ecosystem provider suited to help based on the results. In one example of block 308, the health platform 102 may reference big data 108 and/or a medical professional corpus when matching the patient to the mental health provider. The big data 108 or medical professional corpus may include a table organizing medical professionals according to mental health specialty (e.g., PTSD, depression, etc.), occupation (e.g., psychiatrist, therapist, etc.), education level, years of experience, etc.

FIG. 4 is one example of a system architecture 400. System 400 includes client devices 104(1)-(3), which may receive information from various sources and allows various entities (medical professionals, patients, etc.) access to the health platform 102. For example, client device 104(1) is a patient mental health application, accessible to authorized users (e.g., patients, medical health professionals, etc.) and may transmit user input to the health platform 102, as well as to an automated interactive record 140 and blockchain interface 141 and digital passport platform 150. The client device 104(2) is a mental health awareness application, accessible to a user (e.g., a patient or a prospective patient), and may transmit user input to the health platform 102. The client device 104(3) is a health care provider portal, accessible to health care providers, who may be prospective health care providers or current health care providers associated with the health platform 102. The client device 104(3) may transmit user input to the health platform 102.

The health platform 102 may include an assessment workflow 110, which includes assessment 111 (e.g., medical diagnostics), rules 112 (e.g., invoked by the rules engine), and workflow 113; a treatment workflow 114, which includes a course 115 (e.g., a treatment course, as included with a generated treatment recommendation), a provider 116 (e.g., one of the healthcare providers, such as a therapist, psychiatrist, life coach, etc.) and payment 117; and, a rules engine 118, which includes content 119 and artificial intelligence 120. In one example embodiment, the mental health platform 102 may ingest the data sets from the client devices 104(1), (3), perform the work assessment flow 110, including ingesting the assessment 111 (e.g., static or interactive assessment, with reference to FIG. 2A), applying the rules 112 and treatment workflow 113 to the assessment. Further, the health platform 102 may invoke the treatment workflow 113 (e.g., generating the treatment recommendation), which includes providing a course 115 of treatment and a provider 116 (e.g., a healthcare provider using the healthcare portal 104(3)), and accepting payment 117. An integration layer 121 may integrate the health platform 102 with one of additional applications 130, including medical diagnostics platforms 131(1)-(N). For example, the medical diagnostics platforms 131(1)-(N) (e.g., customer relationship management applications, application program interfaces, marketing and analytic software, etc.), which may include nView ©, HubSpot ©, Valant ©, Segment ©, Azure © notifications connector, etc.

Upon the health platform 102 generating a recommendation based on the ingested data sets, the health platform 102 may transmit the ingested data sets and the generated recommended treatment to big data 108, which includes one or more data corpuses 108(1)-(N), including, e.g., SQL Azure, Azure Functions Collections, Synapse Infrastructure (e.g., Azure Synapse Analytics, etc.), Azure Power BI Dashboards, etc., all of which may be used for big data management, presentation, and analysis. Big data 108 may analyze the data and transmit trends and patterns generated, back to the health platform 102 to be used for generating future treatment recommendations. In one example, an application within big data 108 may determine, based on identifying factors, that a particular treatment is more effective to patients who have similar identifying factors, which may then be used to determine a course of treatment. In one example, the applications may analyze patterns and trends associated with a particular patient over time in relation to outcomes of other patients with similar identifying factors and mental states determined by the medical diagnostics assessment.

The health platform 102 may transmit data to the automated interactive record 140 and the digital passport platform 150. The automated interactive record 140 which may include an application blockchain interface 141, along with records 142, assets 143, events 144, and authentication, authorization, and key management 145. The digital passport platform 150 may include an open source distributed ledger 146, a decentralized identity 147, verifiable credentials 148, and schema management 149. The digital passport 150 and the automated interactive record 140 may transmit date to an interplanetary file system (IPFS) cluster 160 for storing and sharing data between any of the IPFS nodes 161(1)-(N).

FIG. 5 is an example machine learning system 500 used by the rules engine for generating, based on ingested data sets and big data 108.

FIG. 5 is a diagram illustrating an example 500 of training and using a machine learning model in connection with the health platform 102 and rules engine. The machine learning model training and usage described herein may be performed using a machine learning system. The machine learning system may include or may be included in a computing device, a server, a cloud computing environment, and/or the like, such as the health platform 102, described in more detail elsewhere herein.

As shown by reference number 505, a machine learning model may be trained using a set of observations. The set of observations may be obtained from historical data, such as data gathered during the assessment workflow 110 and treatment workflow 113. In some implementations, the machine learning system may receive the set of observations (e.g., as input) from the health platform 102, the client devices 104(1)-(3), external sources 106, and big data 108, as described elsewhere herein.

As shown by reference number 510, the set of observations includes a feature set. The feature set may include a set of variables, and a variable may be referred to as a feature. A specific observation may include a set of variable values (or feature values) corresponding to the set of variables. In some implementations, the machine learning system may determine variables for a set of observations and/or variable values for a specific observation based on input received from the client device 104(1)-(3). For example, the machine learning system may identify a feature set (e.g., one or more features and/or feature values from the medical diagnostics assessment) by extracting the feature set from structured data, by performing natural language processing to extract the feature set from unstructured data, by receiving input from an operator, and/or the like.

As an example, a feature set for a set of observations may include a first feature of PTSD (e.g., determined by the assessment workflow 110), a second feature of gender (e.g., received from the external source 106 or the patient using the client device 104(1), (2)), a third feature of income amount (e.g., received from the external source 106 or the patient using the client device 104(1), (2)), and so on. For a first observation, the first feature may have a value of 3, the second feature may have a value of 5, the third feature may have a value of 6, and so on. These features and feature values are provided as examples, and may differ in other examples. For example, the feature set may include one or more of the following features: mental health, fitness, past injuries, surgeries, diseases, education, marital status, income, alcohol and narcotics history, sexual orientation, race, ethnicity, height, weight, personality, etc.

As shown by reference number 515, the set of observations may be associated with a target variable. The target variable may represent a variable having a numeric value, may represent a variable having a numeric value that falls within a range of values or has some discrete possible values, may represent a variable that is selectable from one of multiple options (e.g., one of multiples classes, classifications, labels, and/or the like), may represent a variable having a Boolean value, and/or the like. A target variable may be associated with a target variable value, and a target variable value may be specific to an observation. In example 500, the target variable is a treatment course, which has a value of 2 for the first observation.

The target variable may represent a value that a machine learning model is being trained to predict, and the feature set may represent the variables that are input to a trained machine learning model to predict a value for the target variable. The set of observations may include target variable values so that the machine learning model can be trained to recognize patterns in the feature set that lead to a target variable value. A machine learning model that is trained to predict a target variable value may be referred to as a supervised learning model.

In some implementations, the machine learning model may be trained on a set of observations that do not include a target variable. This may be referred to as an unsupervised learning model. In this case, the machine learning model may learn patterns from the set of observations without labeling or supervision, and may provide output that indicates such patterns, such as by using clustering and/or association to identify related groups of items within the set of observations.

As shown by reference number 520, the machine learning system may train a machine learning model using the set of observations and using one or more machine learning algorithms, such as a regression algorithm, a decision tree algorithm, a neural network algorithm, a k-nearest neighbor algorithm, a support vector machine algorithm, and/or the like. After training, the machine learning system may store the machine learning model as a trained machine learning model 525 to be used to analyze new observations.

As shown by reference number 530, the machine learning system may apply the trained machine learning model 525 to a new observation, such as by receiving a new observation and inputting the new observation to the trained machine learning model 525. As shown, the new observation may include a first feature of bipolar disorder, a second feature of job occupation, a third feature of marital status, and so on, as an example. The machine learning system may apply the trained machine learning model 525 to the new observation to generate an output (e.g., a result). The type of output may depend on the type of machine learning model and/or the type of machine learning task being performed. For example, the output may include a predicted value of a target variable, such as when supervised learning is employed. Additionally, or alternatively, the output may include information that identifies a cluster to which the new observation belongs, information that indicates a degree of similarity between the new observation and one or more other observations, and/or the like, such as when unsupervised learning is employed.

As an example, the trained machine learning model 525 may predict a value of 5 for the target variable of therapist for the new observation, as shown by reference number 535. Based on this prediction, the machine learning system may provide a first recommendation, may provide output for determination of a first recommendation, may perform a first automated action, may cause a first automated action to be performed (e.g., by instructing another device to perform the automated action), and/or the like. The first recommendation may include, for example, therapist. The first automated action may include, for example, therapist, psychiatrist, life coach, etc.

In some implementations, the trained machine learning model 525 may classify (e.g., cluster) the new observation in a cluster, as shown by reference number 540. The observations within a cluster may have a threshold degree of similarity. As an example, if the machine learning system classifies the new observation in a first cluster (e.g., observations 1, 7, and 9), then the machine learning system may provide a first recommendation, such as the first recommendation described above. For example, observations 7 and 9 may have had user input that includes substantially similar states, identifying factors, and treatment course, as cluster 1. Further, the treatment course provided for observations 7 and 9 may have resulted in improvement to the patient. Additionally, or alternatively, the machine learning system may perform a first automated action and/or may cause a first automated action to be performed (e.g., by instructing another device to perform the automated action) based on classifying the new observation in the first cluster, such as the first automated action described above.

As another example, if the machine learning system were to classify the new observation in a second cluster (e.g., observations 2, 4, and 8), then the machine learning system may provide a second (e.g., different) recommendation (e.g., psychiatrist) and/or may perform or cause performance of a second (e.g., different) automated action. For example, observations 4 and 8 may have had user input that includes substantially similar states, identifying factors, and treatment course, as observation 2. Further, the treatment course provided for observations 4 and 8 may have resulted in improvement to the patient. Each of the clusters and corresponding observations may be stored in big data 108, and retrieved by the machine learning system 500 when recommending a course of treatment.

In some implementations, the recommendation and/or the automated action associated with the new observation may be based on a target variable value having a particular label (e.g., classification, categorization, and/or the like), may be based on whether a target variable value satisfies one or more threshold (e.g., whether the target variable value is greater than a threshold, is less than a threshold, is equal to a threshold, falls within a range of threshold values, and/or the like), may be based on a cluster in which the new observation is classified, and/or the like.

In this way, the machine learning system may apply a rigorous and automated process to recommend a course of treatment based on historical data. The machine learning system enables recognition and/or identification of tens, hundreds, thousands, or millions of features and/or feature values for tens, hundreds, thousands, or millions of observations, thereby increasing accuracy and consistency and reducing the likelihood of injecting conscious and subconscious bias associated with health care providers recommending a court of treatment.

As indicated above, FIG. 5 is provided as an example. Other examples may differ from what is described in connection with FIG. 5.

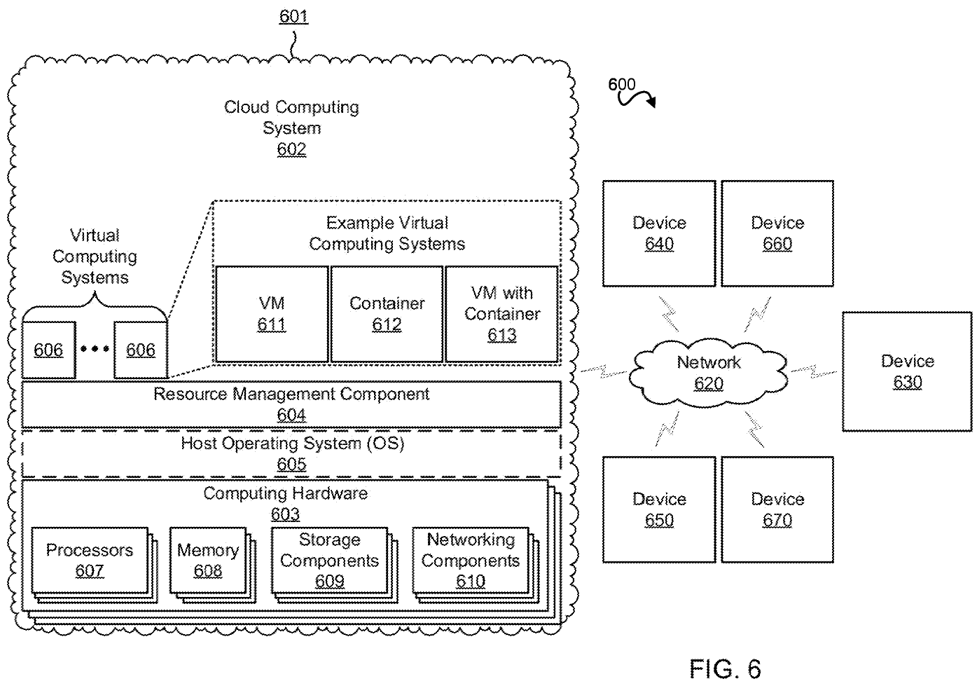

FIG. 6 is a diagram of an example environment 600 in which systems and/or methods described herein may be implemented. As shown in FIG. 6, environment 600 may include a system 601, which may include one or more elements of and/or may execute within a cloud computing system 602. The cloud computing system 602 may include one or more elements 603-613, as described in more detail below. As further shown in FIG. 6, environment 600 may include a network 620, and devices 630, 640, 650, 660, 670, which may be any of client device 104(1)-(3) or other electronic devices capable of communicating over network 620 and hosting one of a health care provider portal (e.g., an application or website), mental health awareness application, and/or patient mental health application, etc. Devices and/or elements of environment 600 may interconnect via wired connections and/or wireless connections.

The cloud computing system 602 includes computing hardware 603, a resource management component 604, a host operating system (OS) 605, and/or one or more virtual computing systems 606. The resource management component 604 may perform virtualization (e.g., abstraction) of computing hardware 603 to create the one or more virtual computing systems 606. Using virtualization, the resource management component 604 enables a single computing device (e.g., a computer, a server, and/or the like) to operate like multiple computing devices, such as by creating multiple isolated virtual computing systems 606 from computing hardware 603 of the single computing device. In this way, computing hardware 603 can operate more efficiently, with lower power consumption, higher reliability, higher availability, higher utilization, greater flexibility, and lower cost than using separate computing devices.

Computing hardware 603 includes hardware and corresponding resources from one or more computing devices. For example, computing hardware 603 may include hardware from a single computing device (e.g., a single server) or from multiple computing devices (e.g., multiple servers), such as multiple computing devices in one or more data centers. As shown, computing hardware 603 may include one or more processors 607, one or more memories 608, one or more storage components 609 (e.g., big data 108), and/or one or more networking components 610 (e.g., that can communicate with the automated interactive record 140, digital passport platform 150, etc.). Examples of a processor, a memory, a storage component, and a networking component (e.g., a communication component) are described elsewhere herein.

The resource management component 604 includes a virtualization application (e.g., executing on hardware, such as computing hardware 603) capable of virtualizing computing hardware 603 to start, stop, and/or manage one or more virtual computing systems 606. For example, the resource management component 604 may include a hypervisor (e.g., a bare-metal or Type 1 hypervisor, a hosted or Type 2 hypervisor, and/or the like) or a virtual machine monitor, such as when the virtual computing systems 606 are virtual machines 611. Additionally, or alternatively, the resource management component 604 may include a container manager, such as when the virtual computing systems 606 are containers 612. In some implementations, the resource management component 604 executes within and/or in coordination with a host operating system 605.

A virtual computing system 606 includes a virtual environment that enables cloud-based execution of operations and/or processes described herein using computing hardware 603. As shown, a virtual computing system 606 may include a virtual machine 611, a container 612, a hybrid environment 613 that includes a virtual machine and a container, and/or the like. A virtual computing system 606 may execute one or more applications using a file system that includes binary files, software libraries, and/or other resources required to execute applications on a guest operating system (e.g., within the virtual computing system 606) or the host operating system 605.

Although the system 601 may include one or more elements 603-613 of the cloud computing system 602, may execute within the cloud computing system 602, and/or may be hosted within the cloud computing system 602, in some implementations, the system 601 may not be cloud-based (e.g., may be implemented outside of a cloud computing system) or may be partially cloud-based. For example, the system 601 may include one or more devices that are not part of the cloud computing system 602, which may include a standalone server or another type of computing device. The system 601 may perform one or more operations and/or processes described in more detail elsewhere herein.

Network 620 includes one or more wired and/or wireless networks. For example, network 620 may include a cellular network, a public land mobile network (PLMN), a local area network (LAN), a wide area network (WAN), a private network, the Internet, and/or the like, and/or a combination of these or other types of networks. The network 620 enables communication among the devices of environment 600.

The number and arrangement of devices and networks shown in FIG. 6 are provided as an example. In practice, there may be additional devices and/or networks, fewer devices and/or networks, different devices and/or networks, or differently arranged devices and/or networks than those shown in FIG. 6. Furthermore, two or more devices shown in FIG. 6 may be implemented within a single device, or a single device shown in FIG. 6 may be implemented as multiple, distributed devices. Additionally, or alternatively, a set of devices (e.g., one or more devices) of environment 600 may perform one or more functions described as being performed by another set of devices of environment 600.

Various embodiments provide a method of using a machine learning model to recommend a course of mental health treatment. The method includes ingesting (for example, by processing, analyzing, etc.), by a health platform, a first data set from a first medical diagnostics assessment of a patient. The health platform ingests a second data set of identifying factors associated with the patient. The method includes applying, by the health platform, to the ingested first and second data sets, a machine learning model and generating, based on the applying the machine learning model to the first and second data sets, a course of mental health treatment. The generated course of mental health treatment is displayed within a graphical user interface.

In a further embodiment of the method above, the method also includes ingesting, by the health platform, a third data set from a second medical diagnostics assessment of the patient and receiving, by the health platform, from a big data source, data related to the third data set. The health platform applies the machine learning model to the third data set and the data from the big data source; and updates the generated course of mental health treatment.

In another embodiment of any one of the methods above, generating, based on the applying the machine learning model to the first and second data sets, the course of mental health treatment, further includes at least one of: recommending a healthcare provider to contact the patient, and suggesting a prescription for the patient.

In a further embodiment of any one of the methods above, generating, based on the applying the machine learning model to the first and second data sets, the course of mental health treatment, is based, at least in part, one or more of: income, age, gender, sexual orientation, race, education level, military experience of the patient.

In another embodiment of any one of the methods above, the course of mental health treatment, includes a type of care and a frequency of care.

In a further embodiment of any one of the methods above, the identifying factors include at least one of: age, gender, medical history, mental health history, fitness, past injuries, past surgeries, past diseases, education, marital status, income, alcohol and narcotics history, sexual orientation, race, ethnicity, height, and weight.

In another embodiment of any one of the methods above, the first data set is stored as vector embeddings. A vector embedding may include at least one numeric value within a range of values. The at least one numeric value may represent a variable that is selectable from one of multiple options. The vector embedding can also include at least one Boolean value.

In a further embodiment of any one of the methods above, the second data set is stored as vector embeddings.

In another embodiment of any one of the methods above, displaying the generated course of mental health treatment further includes displaying reasons for generating the course of mental health treatment.

Further embodiments provide a computer readable medium tangibly encoded with a computer program to recommend a course of mental health treatment. The computer program is executable by a processor to perform actions. The actions include ingesting a first data set from a first medical diagnostics assessment of a patient and ingesting a second data set of identifying factors associated with the patient. The actions also include applying to the ingested first and second data sets, a machine learning model and generating, based on the applying the machine learning model to the first and second data sets, a course of mental health treatment. A graphical user interface is used to display the generated course of mental health treatment.

In another embodiment of the computer readable medium above, the actions further include ingesting a third data set from a second medical diagnostics assessment of the patient; receiving from a big data source, data related to the third data set; and applying the machine learning model to the third data set and the data from the big data source, The actions also include updating the generated course of mental health treatment.

In a further embodiment of any one of the computer readable media above, generating, based on the applying the machine learning model to the first and second data sets, the course of mental health treatment, further includes at least one of: recommending a healthcare provider to contact the patient, and suggesting a prescription for the patient.

In another embodiment of any one of the computer readable media above, the course of mental health treatment, includes a type of care and a frequency of care.

In a further embodiment of any one of the computer readable media above, the first data set and the second data set is stored as vector embeddings.

In another embodiment of any one of the computer readable media above, displaying the generated course of mental health treatment further includes displaying reasons for generating the course of mental health treatment.

In a further embodiment of any one of the computer readable media above, the computer readable medium is a storage medium.

In another embodiment of any one of the computer readable media above, the computer readable medium is a non-transitory computer readable medium (e.g., CD-ROM, RAM, flash memory, etc.).

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method of using a machine learning model to recommend a course of mental health treatment, comprising:
ingesting, by a health platform, a first data set from a first medical diagnostics assessment of a patient;
ingesting, by the health platform, a second data set of identifying factors associated with the patient;
applying, by the health platform, to the ingested first and second data sets, a machine learning model;
generating, based on the applying the machine learning model to the first and second data sets, a course of mental health treatment; and
displaying, within a graphical user interface, the generated course of mental health treatment.

2. The method of claim 1, further comprising:
ingesting, by the health platform, a third data set from a second medical diagnostics assessment of the patient;
receiving, by the health platform, from a big data source, data related to the third data set;
applying, by the health platform, the machine learning model to the third data set and the data from the big data source; and
updating, by the health platform, the generated course of mental health treatment.

3. The method of claim 1, wherein generating, based on the applying the machine learning model to the first and second data sets, the course of mental health treatment, further comprises at least one of: recommending a healthcare provider to contact the patient, and suggesting a prescription for the patient.

4. The method of claim 1, wherein generating, based on the applying the machine learning model to the first and second data sets, the course of mental health treatment, is based, at least in part, one or more of: income, age, gender, sexual orientation, race, education level, military experience of the patient.

5. The method of claim 1, wherein the course of mental health treatment, comprises a type of care and a frequency of care.

6. The method of claim 1, wherein the identifying factors comprise at least one of: age, gender, medical history, mental health history, fitness, past injuries, past surgeries, past diseases, education, marital status, income, alcohol and narcotics history, sexual orientation, race, ethnicity, height, and weight.

7. The method of claim 1, wherein the first data set is stored as vector embeddings.

8. The method of claim 7, wherein a vector embedding comprises at least one numeric value within a range of values.

9. The method of claim 7, wherein the at least one numeric value represents a variable that is selectable from one of multiple options.

10. The method of claim 7, wherein a vector embedding comprises at least one Boolean value.

11. The method of claim 1, wherein the second data set is stored as vector embeddings.

12. The method of claim 1, wherein displaying the generated course of mental health treatment further comprises displaying reasons for generating the course of mental health treatment.

13. A computer readable medium tangibly encoded with a computer program to recommend a course of mental health treatment, the computer program executable by a processor to perform actions comprising:
ingesting a first data set from a first medical diagnostics assessment of a patient;
ingesting a second data set of identifying factors associated with the patient;
applying to the ingested first and second data sets, a machine learning model;
generating, based on the applying the machine learning model to the first and second data sets, a course of mental health treatment; and
displaying, within a graphical user interface, the generated course of mental health treatment.

14. The computer readable medium of claim 13, wherein the actions further comprise:
ingesting a third data set from a second medical diagnostics assessment of the patient;
receiving from a big data source, data related to the third data set;
applying the machine learning model to the third data set and the data from the big data source; and
updating the generated course of mental health treatment.

15. The computer readable medium of claim 13, wherein generating, based on the applying the machine learning model to the first and second data sets, the course of mental health treatment, further comprises at least one of: recommending a healthcare provider to contact the patient, and suggesting a prescription for the patient.

16. The computer readable medium of claim 13, wherein the course of mental health treatment, comprises a type of care and a frequency of care.

17. The computer readable medium of claim 13, wherein the first data set and the second data set is stored as vector embeddings.

18. The computer readable medium of claim 13, wherein displaying the generated course of mental health treatment further comprises displaying reasons for generating the course of mental health treatment.
